(19) United States
(12) Patent Application Publication
(10) Pub. No.: US 2016/0256565 A1
(43) Pub. Date: Sep. 8, 2016

(54) Composite bioabsorbable barrier membranes with sustained dual drug delivery for tissue engineering and guided tissue regeneration

(71) Applicant: Manoj Muthukuru, Morgantown, WV (US)

(72) Inventor: Manoj Muthukuru, Morgantown, WV (US)

(21) Appl. No.: 14/636,139

(22) Filed: Mar. 2, 2015

(51) Int. Cl. A61K 47/48

(52) U.S. Cl. CPC A61K 47/48784; A61K 47/48238

(57) ABSTRACT

In this invention, composite polymeric barrier membrane is developed for clinical applications related to guided tissue regeneration and guided bone regeneration. The compositions of this polymeric membrane have conventional mechanical functions of a barrier membrane and will enable space maintenance and stabilization of the healing surgical wound. In addition, this composite polymeric membrane comprise dynamic bioactive components for dual drug delivery that will enable sustained delivery of drugs, growth factors or relevant molecules for promoting wound healing and tissue regeneration.

### CROSS-REFERENCE TO RELATED APPLICATIONS

This application claims the benefit of earlier filed U.S. Provisional Application No. 61/922,474, filed on Dec. 31, 2013.

### STATEMENT REGARDING FEDERALLY SPONSORED RESEARCH OR DEVELOPMENT

Not applicable

### REFERENCE TO SEQUENCE LISTING

Not applicable

### BACKGROUND OF THE INVENTION

This invention is related to composition of composite polymeric barrier membranes with mechanical properties that facilitate conventional barrier functions along with functional properties or bioactive components that mediate sustained dual drug delivery and the said composition is designed for applications such as tissue engineering, guided tissue regeneration and guided bone regeneration.

From historical perspective, wound dressings were initially fabricated for hygiene and moisture control applications. These dressings evolved to suite specific characteristics of diverse wounds and subsequently were developed into scaffolds for delivering relevant pharmaceutical agents and thereby facilitate wound healing [1]. Advent of health care disciplines such as tissue engineering which were accompanied by biomedical technologies lead to formation of integrative or interdisciplinary principles for technological advances; such bioengineering technologies thereby mediate wound dressing materials emerging into smart, bioactive scaffolds for various plastic surgical procedures and tissue reconstructive specialties. [1-4]. Innovations in nanotechnology have enabled various configurations of particles, fibers, spheres and membranes from natural and synthetic bioresorbable polymers. Integrating polymers with organic-inorganic interphases and exploring environmentally sensitive hydrogels have further facilitated controlled drug delivery systems for regenerative applications in tissue engineering.

Since 1950s, barrier membranes prepared from natural or synthetic biomaterials, either with resorbable or non-resorbable compositions have been used for surgical repair, regeneration or reconstruction of bone in dental, periodontal, maxillofacial/craniofacial and orthopedic surgeries [5]. Guided bone regeneration is surgical technique of utilizing barrier membranes which are usually employed in conjunction with bone grafting materials [6] and principles of guided bone regeneration evolved from guided tissue regeneration, named during 1980s and employed for treating periodontal diseases [7, 8].

The surgical principle involves placing a barrier membrane to protect blood clot and bone grafts (when placed as regenerating materials), maintain secluded space around the bone defect, prevent non-bone forming cells from migrating into this protected surgical space and thereby facilitate bone formation [9-13].

Previous arts (Boyne et al., EP1084720A1 Mar. 2001; Dunn et al., U.S. Pat. No. 5,368,859A November 1994; Geistlich et al., U.S. Pat. No. 5,837,278A November 1998; Geistlich et al., U.S. Pat. No. 6,752,834B2 Jun. 2004; Magnusson et al., U.S. Pat. No. 4,961,707A October 1990) have developed barrier membranes with composite formulations that are usually solid but also with liquid compositions for guided surgical applications in dental and orthopedic specialties.

Post-operative complications associated with guided tissue or guided bone regenerative surgical procedures are usually local in nature and broadly result from infected surgical wounds and excessive inflammatory response and these complications can be further exaggerated due to preexisting systemic medical conditions or diseases and due to aging. Overall, these surgical impediments result in: (A) Delayed wound healing and/or (B) Lack of adequate growth factors which behave as overlapping issues resulting in compromised or ineffective bone regeneration [14, 15]. Consequently, in addition to using barrier membranes, drug delivery methods using carriers or scaffolds were developed for delivering antibiotics, antimicrobials, anti-inflammatory and anti-tissue destructive drugs and also naturally synthesized biological molecules such as growth factors [1, 16] which are aimed to prevent such complications associated with wound healing [17-19] and thereby enable enhanced bone regeneration [20-23].

Relevant experimental research support and suggest effective bone regeneration with drug delivery devices for delivering anti-inflammatory agents or growth factors [24-31] and clinical studies support efficacy with sustained antimicrobial delivery [32] and with growth factor delivery [33-36] resulting in enhanced bone regenerative outcomes. Newer composite polymer membranes [37] and barrier membranes were also designed to enable delivering growth factors [38, 39] or antibiotics [40, 41] thereby facilitating tissue regenerative outcomes.

Previous arts that are relevant to drug delivery systems were designed for concurrent use with barrier membranes in guided tissue regeneration/guided bone regeneration wherein emphasis for bone augmentation through drug delivery systems for therapeutically enhancing wound healing or augmenting tissue regeneration. For instance following representative arts have devised drug delivery systems for transporting and delivering anti-inflammatory agents, growth factors along with angiogenesis inducing agents and genetic material by employing various delivery materials:
(A) Nanofiber based materials as developed by Allcock et al., U.S. Pat. No. 7,235,295B2 Jun. 2007 and Chu et al., U.S. Pat. No. 6,689,374B2 Feb. 2004
(B) Biodegradable composites interfaces with microparticles or nanoparticles were developed by Ashammakhi et al., US20060002979A1 Jan. 2006, CHOI et al., US20130045266A1 Feb. 2013, Elly et al., WO2001035932A2 May 2001 Lynch et al., U.S. Pat. No. 8,114,841B2 Feb. 2012
(C) Lipids, ester based materials and light polymerization systems for drug delivery systems were explored by Borbely, U.S. Pat. No. 6,326,511B1 Dec. 2001 Emanuel et al., 8877242B2 Nov. 2014 Domb et al., EP0605536A4 Jul. 1994
(D) Solvents, injectable liquid interfaces and hydrogels were employed for drug delivery by DDS Damani et al., U.S. Pat. No. 5,447,725A September 1995 U.S. Pat. No. 6,123,957A September 2000 Martin et al., US20140275325A1 Sep. 2014 Noff et al., U.S. Pat. No. 6,682,760B2 Jan. 2004 YOUNG et al., U.S. Pat. No. 8,252,851B2 Aug. 2012 Jernberg, DDS Coady et al., U.S. Pat. No. 8,633,296B1 Jan. 2014
(E) Bone matrices and scaffolds as drug delivery systems were developed by DDS (growth factors, angiogenesis) INANç et al., WO2011030185A1 May 2011 Evans et al., U.S. Pat. No. 8,685,432B2 Apr. 2014 Harris et al., U.S. Pat. No. 6,797,738B2 Sep. 2004 Hsieh et al., US20060149392 A1 Jul. 2006 Shea et al., US20060002978A1 Jan. 2006 Fukushima et al., US20110257094A1 Oct. 2011

The above referenced representative arts are innovations in biomedical research for developing drug delivery systems and have tissue engineering applications and usage in surgical procedures such as guided tissue regeneration. In comparison to these above referenced arts related to local drug delivery through innovative scaffolds and carriers systems, efforts or innovations in developing barrier membranes that would be equipped or capable of delivering drugs, growth factors and antimicrobial agents are relatively sparse and representative arts that have focused on developing barrier membranes into drug delivery devices are CN10190538 April 2013; Chung et al., WO2012144778A9 Jan. 2013; Jernberg, U.S. Pat. No. 5,197,882A March 1993; Liu, U.S. Pat. No. 6,300,315B1 Oct. 2001.

Effective surgical outcomes with respect to bone augmentation procedures as briefly discussed are negatively regulated by concurring and overlapping complications that result in poor wound healing and ineffective bone regeneration and current medical and dental practices associated with guided tissue regeneration and guided bone regeneration routinely encounter adverse outcomes necessitating additional surgical interventions and thereby placing increased health care burden which is of significant concern [42, 43],

From the foregoing introduction on historical perspective with guided tissue regeneration and guided bone regeneration, the fundamental principle for these guided procedures relies on stabilizing blood clot and bone forming matrix at the bony defect and the mechanical properties related to resilience and stability of the barrier membrane play crucial role during initial phases of wound healing which facilitate bone regeneration.

Moreover, barrier membranes are the most proximate biomaterials to the areas that are surgically incised and sutured that undergo wound healing and these healing sites are vulnerable, mediating access to infections which could result in wound healing complications and poor bone augmentation.

The above referenced arts are innovative technologies for drug delivery methods but lack barrier functions necessary for wound stabilization and protection. As a consequence, current methods in guided tissue regeneration employ barrier membranes in addition to these drug delivery devices. While barrier membranes have been developed as drug delivery devices through previous arts and referenced, innovations in barrier membrane technology as more comprehensive and effective biomaterials would be necessary to target and control broader range of post-surgical complications.

In this present invention, compositions for barrier membrane are disclosed which is composite polymeric biologically active barrier membrane that will not only provide mechanical support during wound healing but will also function as dual drug delivery system for delivering antibiotics, antimicrobials agents, anti-inflammatory, anti-tissue destructive drugs and growth factors. The claimed invention differs from what currently exists as this improved technology is equipped to handle wider spectrum of unfavorable events that complicate wound healing and bone regeneration through dynamic dual drug delivery system for enhanced regenerative outcomes. By incorporating functions of conventional barrier membranes and innovative dual drug delivery aspects, the claimed invention i.e., composite polymeric barrier membrane can facilitate curtailing additional biomaterial carriers or scaffolds that have been currently used for delivery drug in addition to employing barrier membranes in guided tissue or guided bone regenerations and thereby enable to reduce inventory and financial burden while enhancing favorable outcomes with tissue engineering applications in health care.

### BRIEF SUMMARY OF THE INVENTION

This invention discloses the compositions for composite polymeric barrier membranes and their dynamic and functional applications with dual drug delivery in tissue engineering, guided tissue regeneration and guided bone regeneration.

In one aspect, the invention discloses the compositions of the barrier membrane consisting of (1) two peripheral components composed of fibrous polymers and (2) a core intermediate component composed of hydrogel microspheres. The two peripheral fibrous polymeric components enclose the central core hydrogel microsphere component and the said components are the preferred embodiments of the invention.

In one aspect, the invention discloses the bioactive components of the barrier membrane wherein, two distinct or identical drugs or molecules relevant to the biomedical applications are incorporated into the core intermediate hydrogel microsphere component and into one of the fibrous polymeric peripheral component.

In one aspect, the invention discloses the said embodiments are distinctly crosslinked for biomechanical functions, wherein the barrier membrane provides mechanical barrier support through the non-drug incorporating fibrous polymeric component; further, the invention discloses the said embodiments independently regulate the kinetics of drug release from core hydrogel component and drug incorporated fibrous polymeric peripheral component.

From the foregoing disclosure of the preferred embodiments, composite polymeric barrier membranes will enable broader kinetic range of sustained dual drug delivery for wide spectrum of bioactive molecules through stimuli-responsive compositions and with regulated crosslinking aspects which thereby facilitate diversity in tissue engineering applications as disclosed in this invention.

### BRIEF DESCRIPTION OF THE SEVERAL VIEWS OF THE DRAWING

FIG. 1 Schematic of the compositions demonstrating Outer fibrous component, Core Intermediate Hydrogel Component and Inner Fibrous Component with their functions labelled as Mechanical Barrier, Drug Delivery A and Drug Delivery B respectively.

FIG. 2 Light microscopic imaging of uncrosslinked gelatin microspheres

FIG. 3 Light microscopic imaging of crosslinked, doxycycline incorporated gelatin microspheres

FIG. 4 Electron microscopic imaging of uncrosslinked gelatin microspheres

FIG. 5 Electron microscopic imaging of crosslinked, doxycycline incorporated gelatin microspheres

FIG. 6 Light microscope imaging of collagen bilayer embedding gelatin microspheres

FIG. 7 Kinetics of doxycycline release from gelatin microspheres at three different crosslinking concentrations

FIG. 8 Kinetics of dexamethasone release from inner fibrous component of collagen bilayer at three different crosslinking concentrations

FIG. 9 Alkaline phosphatase assay on osteoblast—monocyte co-cultures

FIG. 10 Bone staining (mineralized nodule formation by von kossa method) of osteoblasts cultured on drug free collagen bilayer—microsphere composite

FIG. 11 Bone staining (mineralized nodule formation by von kossa method) of osteoblasts cultured on collagen bilayer—microsphere composite with doxycycline alone

FIG. 12 Bone staining (mineralized nodule formation by von kossa method) of osteoblasts cultured on collagen bilayer—microsphere composite with dexamethasone alone

FIG. 13 Bone staining (mineralized nodule formation by von kossa method) of osteoblasts cultured on collagen bilayer—microsphere composite with doxycycline—dexamethasone

### DETAILED DESCRIPTION OF THE INVENTION

The following detailed description of the invention is presentation of general principles of the embodiments of the invention and should not become limiting from the context of the terminology or choice and source of materials, chemicals or reagents that are selected to describe the underlying technology through the preferred embodiments as detailed in the following, in non-exhaustive manner:

Type I collagen from bovine source is used in the embodiments for fabricating fibrous component of the bilayer collagen membrane and can be replaced by any other type of collagen, collagenous, non-collagenous materials and derived from any source.

Gelatin type B is used in the in the embodiments of core intermediate component of microsphere and can replaced by any other natural or synthetic material for composing.

The term microsphere is used in non-confining context for drug delivery and can be replaced by nanosphere compositions or any other drug delivery device(s) manufactured from any other materials and source.

Inflammatory response during wound healing, more so when superimposed by infections, result in activation of tissue catabolic enzymes [44] and could negatively affect tissue regeneration [45] Along with antimicrobial bacteriostatic functions doxycycline at sub-antimicrobial doses is also capable of inhibiting tissue destructive enzymes MMPs [46] and dexamethasone apart from anti-inflammatory functions also possesses bone regenerative properties [47] and thereby doxycycline and dexamethasone are used as drugs to demonstrate the workings and the flexible and dynamic technology of said dual drug delivery system, and the term “drug” is used in non-limiting manner and refers to any growth factor or biological or chemical or pharmaceutical molecule used to improve wound healing and/or bone formation.

Relationships of the selected embodiments are as follows: Bone regeneration and guided tissue regeneration or guided bone regeneration surgical concepts are used to describe the compositions and the principle of this invention. Thereby, doxycycline which has known antimicrobial role, which also possesses anti-collagenase or anti-tissue destructive functions during inflammation, at specific pharmacological concentrations is selected and incorporated in the embodiment of microspheres. Crosslinking the microspheres can be regulated in such a manner that the release of the incorporated drug, i.e., doxycycline in this said example can be controlled to the extent that it becomes actively available in the wound at certain inflammatory threshold producing inflammatory enzymes or tissue destructive collagenase enzymes to degrade the composition of microspheres, which is gelatin herein, in this non confining illustration. Dexamethasone, as the second drug to be incorporated into the embodiment of the inner aspect or the wound facing component of the collagen bilayer and to be delivered through this dual drug delivery system is selected for its bone augmenting functions apart from anti-inflammatory properties and to exemplify how appropriate selection of the two drugs could have additive or synergistic effects in the overall regenerative outcome of bone, as non-limiting illustration. The extent of crosslinking and thereby the release kinetics of this second drug, i.e., dexamethasone through the collagen content of the inner aspect or the wound facing aspect of the collagen bilayer is independent in terms of the crosslinking and release kinetics from that of the microspheres, the embodiment that incorporates doxycycline, the first drug in this example and the crosslinking of these two drug releasing embodiments are further independent of the nature of crosslinking of the outer layer or non-wound facing side, the third embodiment whose function remains primarily of a barrier, i.e. to exclude adjoining non-bone tissue cells and being protective of the surgical space and the underlying embodiments of this invention. The said components encompass, the preferred embodiments of the invention, i.e. bi-layered collagen barrier membrane sandwiching microspheres and facilitate mechanical barrier functions and mediate functions of dual drug delivery device.

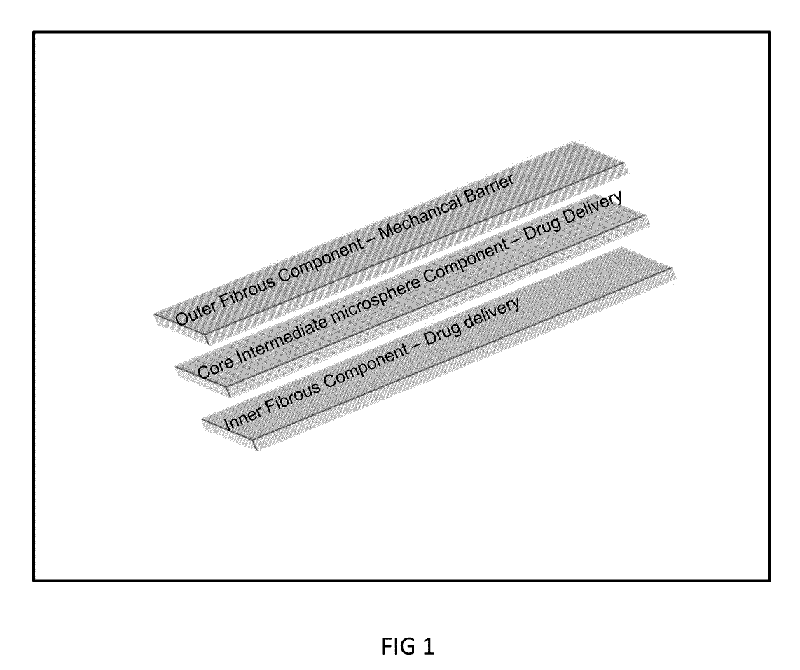

Crosslinking agent can be any physical or chemical agent for crosslinking molecules for the purpose of regulating the mechanical properties of mediating barrier functions of the composite barrier membrane and for regulating kinetics of drug delivery for sustained and controlled drug delivery and therefore become suitable for any drug to be incorporated and delivered and to therapeutically benefit diverse clinical or surgical situations. Following Examples 1-14 are illustrative of the invention, as schematically shown (FIG. 1)

### Example 1

### Preparation of Gelatin Microspheres

Aqueous gelatin solution (15% w/v) was prepared by dissolving bovine type-B gelatin in water. Aqueous gelatin solution was heated to 60° C. and subsequently 10 ml was added to soya oil (preheated to 60° C.). Aqueous-oil phases were placed on a heated magnetic stirrer and emulsified for 5 min. under rotating at 400 rpm. The emulsion was rapidly cooled to 5° C. with continuous stirring for 30 min. to induce gelation. The microspheres were subject to dehydration in acetone (150 ml) precooled to 5° C. with continued stirring for additional 30 min. The microspheres were collected from the suspension through filtration and were washed several times in acetone to remove any residual oil and subsequently vacuum dried for minimum period of 48 hours.

### Example 2

### Crosslinking Gelatin Microspheres

Gelatin microspheres were crosslinked by using 1-ethyl-3-(3-dimethylaminopropyl) carbodiimide hydrochloride (EDC) as crosslinking agent. Briefly, microspheres (500 mg) were suspended in 25 ml of acetone:water (4:1) containing 50 mM of EDC and constantly stirred on magnetic stirrer for 24 hours. The entire process of crosslinking was carried out at 5° C. The reaction mixture was centrifuged at 4000 rpm for 5 min. and the supernatant was discarded. The crosslinking reaction was quenched by suspending microspheres in a 0.1 M Na₂HPO₄ and 2M NaCl solution for 1 hour with moderate stirring on magnetic stirrer. Microspheres were then filtered and extensively washed with deionized water for 20 min. and were sequentially dehydrated with 50% and absolute acetone. The crosslinked microspheres were collected and vacuum dried for at least 48 hours.

### Example 3

### Preparation of Doxycycline Loaded Gelatin Microspheres (Method A)

4% doxycycline solution was prepared in water through emulsification using sunflower oil with 1% (w/w) Span 80 at room temperature and under stirring for 1 hour and 10 ml of doxycycline containing solution was used to prepare gelatin microspheres as described in Example 1

### Example 4

### Preparation of Doxycycline Loaded Gelatin Microspheres (Method B)

Swelling characteristics of crosslinked microspheres were used to entrap the doxycycline hyclate into the microspheres. Preformed empty microspheres (500 mg) were wetted to swell with 2.5 ml of deionized water containing 500 mg of doxycycline hyclate and vortexed for 30 min. The drug loaded microspheres were then washed with alcohol and then vacuum dried. To determine the drug content, known amount of drug loaded microspheres were solubilized in 1 ml of 2N HCl at 60° C. for 2 h and then filtered. The filtrate was analyzed for doxycycline content in microspheres through spectrophotometric methods (OD_{267 nm})

### Example 5

### Characterizing Gelatin Microparticles and Determining Particle Size

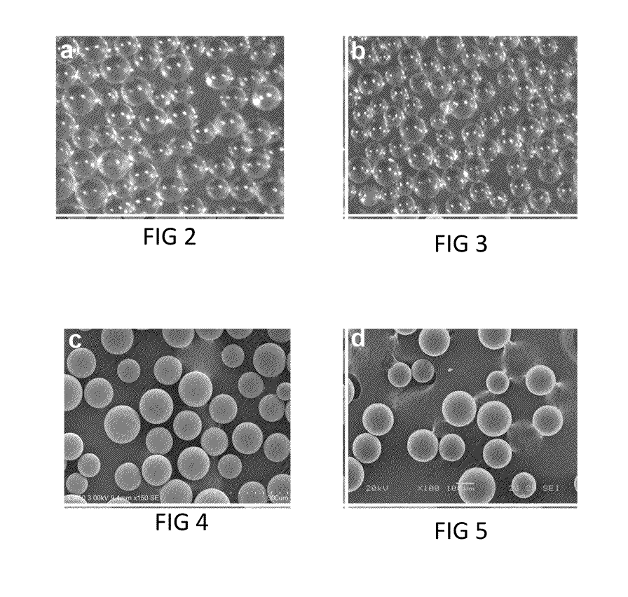

Morphology of gelatin microspheres was determined by light and scanning electron microscopy (SCM). For SCM, samples were mounted on aluminum mounts which was sputter coated with gold and captured at 300 mm focal length using isopropanol as non-reacting dispersion medium and the samples were in suspension until completion of analysis. Particle size ranged from 110-150 micrometers before crosslinking and 90-135 micrometers after crosslinking and presented in FIG. 2-FIG. 5.

### Example 6

### Determining Drug Loading

Drug loaded gelatin microspheres equivalent to 10 mg of doxycycline were digested with 50 ml phosphate buffered solution (PBS; pH 7.4) at room temperature for 12 hours and filtered and analyzed by HPLC to determine the amount of doxycycline present in the microspheres. Drug loading in microspheres was estimated by using the formula: L=Qm/Wm×100 (L is the percentage loading of microspheres, Qm the quantity of doxycycline present in Wm grams of microspheres).

The amount of doxycycline encapsulated in the microspheres was determined using the formula: E=Qp/Qt×100 (E is the percentage encapsulation of microspheres, Qp is the quantity of drug encapsulated in microspheres (g) and Qt is the quantity of doxycycline added for encapsulation (g))

### Example 7

### Determination of Entrapment Efficiency

The swelling behavior of the cross-linked microspheres was used to entrap the doxycycline into the microspheres. Pre-formed empty microspheres (500 mg) were wetted to swell with 2.5 ml of deionized water containing 500 mg of doxycycline and vortexed for 60 min. The drug-loaded microspheres were then washed twice with alcohol and then vacuum-dried. Drug loaded microspheres equivalent to 10 mg of doxycycline were dissolved in dilute acidic condition for 15 min. and the supernatant from the centrifuged solution analyzed. The percentage of entrapment efficiency was calculated by using the following formula: % Entrapment Efficiency=(Theoretical Drug−Released drug)/Theoretical Drug×100. On the basis of Examples 5-7, entrapment efficiency was determined to range between 7-10 mg/100 mg of microspheres (data obtained from 5 samplings)

### Example 8

### Preparation of Dexamethasone—Collagen Conjugates

Type I collagen (5 g) previously extracted from bovine achilles tendon was added to 2 liters of water (pH adjusted to 2.5 with 0.1% (v/v) 12 M HCl) and pH of solubilized collagen was adjusted to 9.0 with NaOH solution. 2% succinic anhydride solution was prepared in acetone and was gradually added to the collagen solution with pH maintained at 9.0 through additional NaOH. Succinylated collagen was precipitated by reducing pH to 4.2. Precipitated succinylated collagen was washed in water (pH 4.2) at 25° C. aseptically. Membranes were cross-linked by hydro-thermal method in a vacuum oven at 100° C. for 24 hours. Dexamethasone (solubilized) was mixed with 10% (w/v) polyvinylpyrrolidone (PVP) and gradually incorporated into collagen by allowing to diffuse at concentration of 0.2 mg/cm². The collagen in the form of sponge after drug incorporation was allowed to air dry and was covered with collagen membrane (please refer to Example 9). Collagen layers were adhered through gentle pressure to formulate multilayered structure.

### Example 9

### Casting Composite Bilayer Collagen Membrane

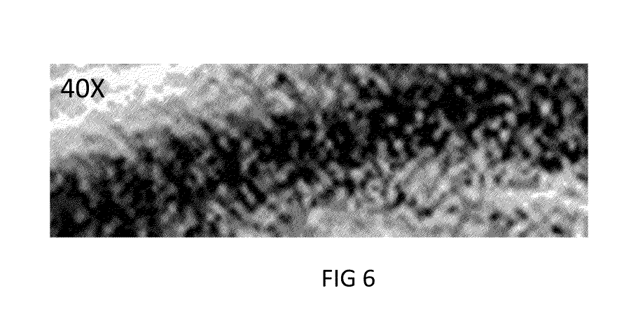

Collagen solution obtained by dissolving in Milli-Q water (pH 3.0) was homogenized before casting. The prefixed volume of the collagen solution was casted on Teflon plate and allowed to semi-dry at room temperature (25° C.). Second layer of collagen solution was cast over the semi-dried primary layer and the process repeated to obtain multi-layered collagen scaffold. Drug encapsulated collagen (from Example 8) was incorporated as intermediate layers and the matrix was freeze dried. Dexamethasone loaded collagen bilayer was prepared to encapsulate doxycycline loaded gelatin microspheres as shown in FIG. 6. Additionally, drug free, doxycycline only or dexamethasone only scaffolds were prepared as controls for bone assays as elaborated in Examples 12-14.

### Example 10

### In Vitro Release Kinetics of Drugs from Composite Bilayer Collagen Membrane

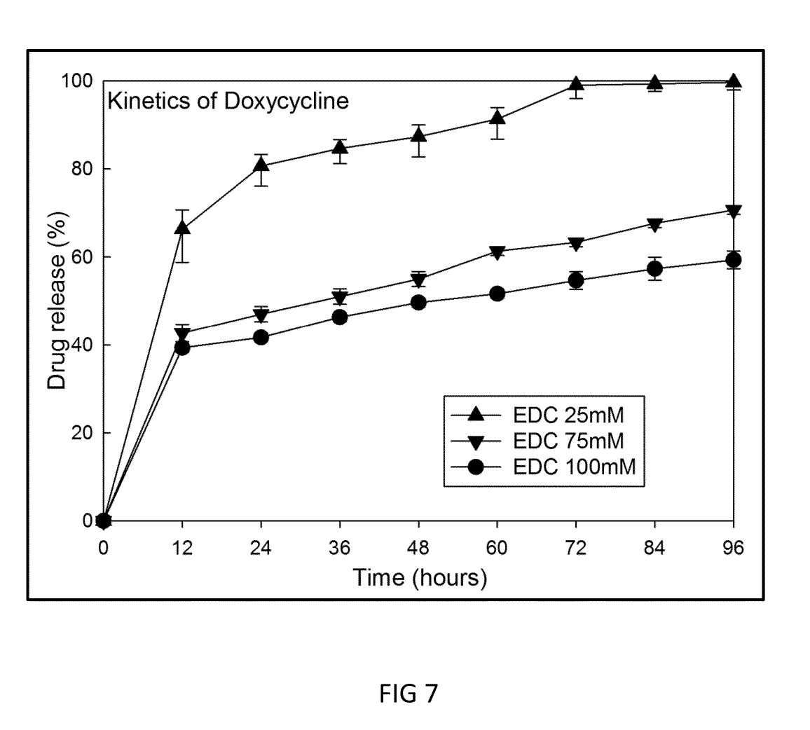

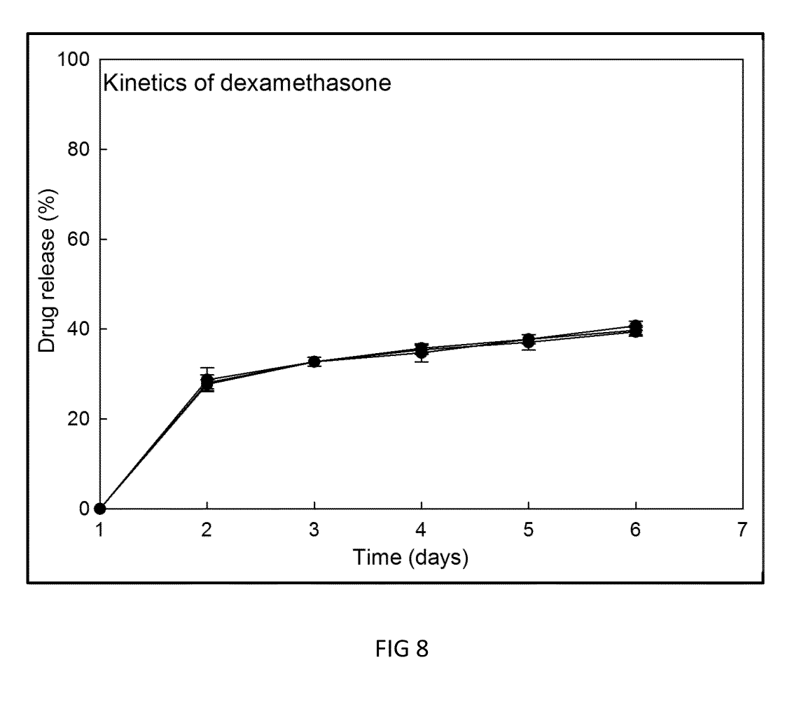

Drug loaded microspheres (50 mg) were poured into a dialysis tube and then placed into 40 ml of physiological synthetic serum electrolyte solution (pH 7.4) at 37° C., with constant shaking. Aliquots of 5 ml were withdrawn at specific time intervals and assayed for the drug content through spectrophotometry (OD_{267 nm}) In vitro release assays were also performed through Franz-Type diffusion cell Apparatus at 37° C. in PBS (pH 7.4). Drug release from collagen bilayer samples were collected at predetermined time intervals and total volume maintained with fresh PBS buffer and pattern of drug release from the bilayer was performed in Franz-type diffusion cell at 37° C. as shown in FIG. 7 and FIG. 8.

### Example 11

### Water Uptake Study for Composite Bilayer Collagen Membrane

Water uptake was calculated by taking gelatin microsphere incorporated bilayer collagen membrane of prefixed dimension and dry weight was measured before soaking in PBS. The samples were removed from PBS buffer at regular time intervals and weighed. The sample mass change resulting from water uptake was calculated by using the formula: % Δm=(m₁−m₀−mₜ)×100 (m₀ and mₜ are the mass of dry and wet samples respectively).

### Example 12

### Bone Cell Cultures and Cytotoxicity Assay

Human primary bone forming cells, namely, osteoblasts were cultured in alpha—minimal essential medium supplemented with 100 units/ml penicillin, 100 microgram/ml streptomycin, 25 microgram/ml fungizone, 2 mM L-glutamine along with 10% heat inactivated fetal bovine serum as described previously [48, 49] in a humidified atmosphere with 5% CO₂ for 7 days. The culture medium was changed every two days and after the cells became confluent, the monolayers were separated using 1× Trypsin—EDTA solution and the cells were expanded in 25 mm² polystyrene flasks. The cultured cells were subsequently grown on composite membranes with or without drug(s) incorporated to determine cytotoxicity by toluidine blue staining and MTT assay and the viability of cells were greater than 95%.

### Example 13

### Osteoblasts—Monocytes Co-Culture to Simulate Inflammatory Response and Alkaline Phosphates Assay

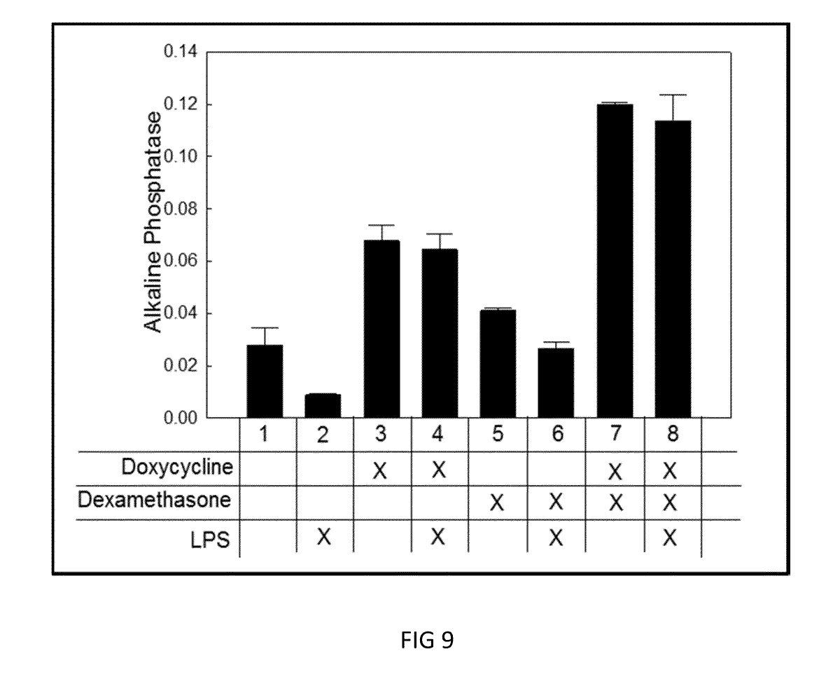

Osteoblasts were cultured as described in Example 12 along with monocytes isolated as previously described [50-53] and osteoblast—monocyte co-cultures were stimulated with 1 microgram/ml lipopolysaccharide (LPS) to induce inflammatory response and simulate adverse wound healing and grown on A, B, C or D compositions listed in Example 14 and alkaline phosphatase assayed as shown in FIG. 9.

### Example 14

### Osteoblast Cultures on Composite Barrier Membranes and Mineralized Bone Nodule Formation Assay

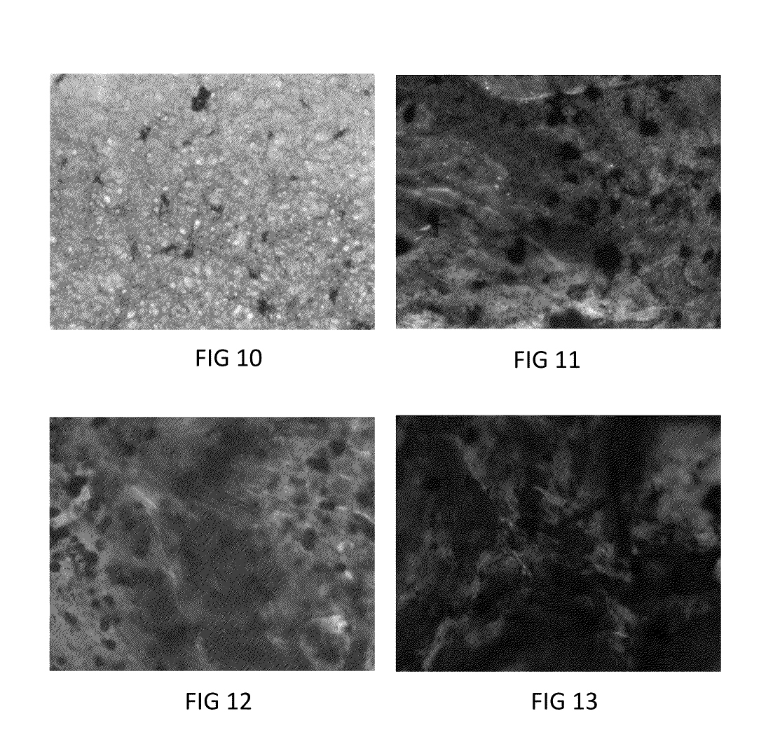

Osteoblasts were cultured as described in Example 12 on one of the four composite compositions: (A) Empty composites—no drug; (B) Composites with doxycycline in the bioactive microsphere hydrogel component; (C) Composites with dexamethasone in the bioactive collagen bilayer; and (D) Composites with doxycycline and dexamethasone in two bioactive components of the composite membrane and in vitro mineralized nodule formation was analyzed as previously reported [49, 50] and illustrated in (FIG. 10-FIG. 13).

Brief discussion and relevance of illustrated Examples (1-14): Schematic representation of compositions demonstrating outer and inner fibrous components and core intermediate microsphere component with their functions labelled as mechanical barrier and drug delivery is presented in FIG. 1. Light and SEM microscopic imaging of un-crosslinked gelatin microspheres or crosslinked with doxycycline demonstrate uniform structural constitution (FIG. 2-FIG. 5) for uniform drug release characteristics and moreover, drug loading and release kinetics can be effectively regulated by altered size characteristics for appropriate drug delivery applications. Moreover, kinetics of doxycycline release from gelatin microspheres were conducted at three different EDC crosslinking concentrations and the rate of diffusion is characteristically reduced with increased crosslinking (FIG. 7) and similarly kinetics of dexamethasone released from fibrous collagen component is shown in FIG. 8. Gelatin as an example here demonstrates flexible constitution for composing microspheres and being a solid particle exhibits surface diffusion. These microparticles can be substituted with hallow spheres having double-drug diffusion aspects with suitable hydrogels and thereby alter the characteristics of zero-order kinetics, as demonstrated in this illustration (FIG. 6) to first-order kinetics and thereby alter the delivery methods from sustained delivery to controlled delivery systems. For effective regeneration of tissues with low renewal rates such as bone, controlled delivery systems would be most suitable as the drug release is regulated in relation to the available concentration in the wound environment. This is also essential to prevent toxicity as has been reported with growth factors such as bone morphogenic proteins delivered using collagen sponges as carriers but without sustained or controlled drug delivery aspects.

Alkaline phosphatase and bone nodule formation assays (Examples 12-14) provide clinically significant information: Alkaline phosphatase assay was conducted by co-culturing osteoblasts with monocytes (1:5) and inflammatory environment was induced by adding 1 microgram/ml LPS (groups 2, 4, 6 and 8) which downmodulated alkaline phosphatase expression (group 2) compared to unstimulated control (group 1). Doxycycline was more potent (group 3) relative to dexamethasone (group 5) in inducing alkaline phosphatase but combined doxycycline and dexamethasone additively upregulated alkaline phosphatase expression (group 7) relative to unstimulated control (group 1). Doxycycline was more resistant relative to dexamethasone for down modulation of alkaline phosphatase when LPS was added and combined doxycycline and dexamethasone suggest little alteration of alkaline phosphatase with LPS (group 8).

For mineralized bone nodule assay, drug free collagen bilayer—microsphere composite membrane served as a negative control (FIG. 10) and in comparison, collagen bilayer—microsphere composite with doxycycline alone (FIG. 11) or dexamethasone alone (FIG. 12) demonstrate increased nodular density and by combining the drugs, i.e. doxycycline and dexamethasone in the composite membrane (FIG. 13), von kossa staining suggests additive or a synergistic effect with significantly intense bone nodule formation with dual drug delivery system. The choices of using these drugs were to illustrate combinatorial effects as in clinical setting, dual drug delivery system such as the compositions of this invention can be utilized for delivering growth factors along with tissue conditioning agents aimed to regulate inflammatory response and thereby reduce collage induced catabolic events. The delivery can be independently regulated in this system and tailored to specific healing events that occur post-operatively.

The compositions of this invention have been employed from routine biological and biocompatible materials, as preferred embodiments, to demonstrate the flexibility and dynamic nature of the invention, i.e., composite bilayer barrier membrane. Obtaining additive or synergistic drug effects with appropriately chosen pharmacological agents and growth factors will have tremendous regenerative potential as illustrated in Examples. Also, for instance, if both microspheres and collagen, i.e. the embodiments of the dual drug delivery system were composed of a single drug, the release kinetics and diffusion aspects can be regulated in more diverse manners for applications to enhance wound healing as one bioactive delivery can be engineered for rapid release and the other system can be engineered for sustained delivery or even for controlled delivery as mentioned above. In clinical or surgical settings, efficient early wound management to prevent immediate post-operative complications is essential but also prolonged delivery through second bioactive system can facilitate controlled delivery directed towards tissue regeneration.

Thermal and pH sensitive polymers when incorporated into this composite system will enable smart regulation of inflammatory events as polymeric hydrogels are extremely sensitive to minute environmental changes. When incorporated, drug delivery systems can be entirely engineered to sense these physical changes and appropriately mediate drug delivery functions. Polymeric brushes can be infused to create on-off switches for sensing and regulating these physical parameters that are cardinal events in the inflammatory microenvironment.

Regulating inflammatory response and preventing the associated tissue destructive events along with sustaining favorable growth environment are crucial for overall regenerative outcomes and barrier membranes through innovative biomaterial technologies have fundamental role to safeguard both early and late healing events. Composite polymeric barrier system, the composition of this invention has robust biomechanical properties that will also ensure space maintenance and wound stabilization which is the historic principle for using barrier membranes in guided surgeries while being designed as dynamic dual drug delivery system for guided tissue regeneration and bioengineering applications, for these biomedical devices guide enormous events that occur in biology and enable regulating wide array of healing events and undesirable complications and ensure favorable clinical and tissue regenerative outcomes.

In this invention, innovative tissue engineering strategies have been presented as an illustration to demonstrate the dynamic and versatile nature of the compositions, the preferred embodiments of composite polymeric barrier membrane.

### PATENT REFERENCES

Emanuel, N., Neuman, M., Barak, S., 8877242B2 Nov. 2014. Sustained-release drug carrier composition. Vol., ed.̂eds. Google Patents.
CN10190538 April 2013. Growth factor-loaded collagen group composite material as well as preparation method and application thereof. Vol., ed.̂eds.
Domb, A. J., Maniar, M., EP0605536A4 Jul. 1994. Fatty acid terminated polyanhydrides. Vol., ed.̂eds. Google Patents.
Martin, D. P., Skraly, F. A., Williams, S. F., EP1042388 B1 Nov. 2006. Polyhydroxyalkanoate compositions having controlled degradation rates. Vol., ed.̂eds. Google Patents.
Boyne, P. J., Geistlich, P., Schlösser, L., EP1084720A1 Mar. 2001. Use of a collagen membrane in a method of protecting spinal area. Vol., ed.̂eds. Google Patents.
Magnusson, I., Batich, C., U.S. Pat. No. 4,961,707A October 1990. Guided periodontal tissue regeneration. Vol., ed.̂eds. Google Patents.
Jernberg, G. R., U.S. Pat. No. 5,197,882A March 1993. Periodontal barrier and method for aiding periodontal tissue regeneration agents. Vol., ed.̂eds. Google Patents.
Dunn, R. L., et al., U.S. Pat. No. 5,368,859A November 1994. Biodegradable system for regenerating the periodontium. Vol., ed.̂eds. Google Patents.
Damani, N.C., Mohl, D. C., Singer, R. E., U.S. Pat. No. 5,447,725A September 1995. Methods for aiding periodontal tissue regeneration. Vol., ed.̂eds. Google Patents.
Geistlich, P., Eckmayer, Z., Boyne, P., U.S. Pat. No. 5,837,278A November 1998. Collagen membranes for grafting and fibers. Vol., ed.̂eds. Google Patents.
Jernberg, G. R., U.S. Pat. No. 6,123,957A September 2000. Combining at least one tissue regenerative agent with at least one cellular recognition agent(cra) to form a therapeutic treatment composition and applying it to a periodontal treatment site; cra increases tissue regeneration. Vol., ed.̂eds. Google Patents.
Liu, S. T., U.S. Pat. No. 6,300,315B1 Oct. 2001. Mineralized collagen membrane and method of making same. Vol., ed.̂eds. Google Patents.
Borbely, J., U.S. Pat. No. 6,326,511B1 Dec. 2001. Polymeric product. Vol., ed.̂eds. Google Patents.
Noff, M., Pitaru, S., U.S. Pat. No. 6,682,760B2 Jan. 2004. Cross-linked collagen matrices and methods for their preparation. Vol., ed.̂eds. Google Patents.
Chu, B., et al., U.S. Pat. No. 6,689,374B2 Feb. 2004. Biodegradable and/or bioabsorbable fibrous articles and methods for using the articles for medical applications. Vol., ed.̂eds. Google Patents.
Geistlich, P., Eckmayer, Z., Schlösser, L., U.S. Pat. No. 6,752,834B2 Jun. 2004. Bone or cartilage reconstruction; collagen ii open spongelike texture matrix with barrier layer. Vol., ed.̂eds. Google Patents.
Harris, L., Mooney, D. J., Shea, L., U.S. Pat. No. 6,797,738B2 Sep. 2004. Open pore biodegradable matrices. Vol., ed.̂eds. Google Patents.
Allcock, H. R., et al., U.S. Pat. No. 7,235,295B2 Jun. 2007. Polymeric nanofibers for tissue engineering and drug delivery. Vol., ed.̂eds. Google Patents.
Martin, D. P., Skraly, F. A., U.S. Pat. No. 8,039,237 B2 Oct. 2011. Bioabsorbable polymer containing 2-hydroxyacid monomers. Vol., ed.̂eds. Google Patents.
Calhoun, C. J., Holmes, R. E., Cornwall, G. B., U.S. Pat. No. 8,048,444 B2 Nov. 2011. Applying to the implant a bioresorbable membrane made of lactide polymers or lactone copolymers. Vol., ed.̂eds. Google Patents.
Lynch, S. E., Nevins, M., Simion, M., U.S. Pat. No. 8,114,841B2 Feb. 2012. Maxillofacial bone augmentation using rhPDGF-BB and a biocompatible matrix. Vol., ed.̂eds. Google Patents.
YOUNG, A. M., HO, S. M., U.S. Pat. No. 8,252,851 B2 Aug. 2012. Formulations and composites with reactive fillers. Vol., ed.̂eds. Google Patents.
Kuo, Y., Violante, M. R., U.S. Pat. No. 8,603,051 B2 Dec. 2013. Implantable drug delivery devices. Vol., ed.̂eds. Google Patents.
Coady, D. J., et al., U.S. Pat. No. 8,633,296B1 Jan. 2014. Composite hydrogels for delivery of biologically active materials. Vol., ed.̂eds. Google Patents.
Evans, B. G., et al., U.S. Pat. No. 8,685,432B2 Apr. 2014. Controlled release tissue graft combination biomaterials. Vol., ed.̂eds. Google Patents.
Hsieh, K. H., et al., US20060149392 A1 Jul. 2006. Biomaterials for guided tissue regeneration and drug delivery. Vol., ed.̂eds. Google Patents.
KOOB, T., Daniel, J., Spencer, R., US20130202676A1 Aug. 2013. Cross-linked dehydrated placental tissue grafts and methods for making and using the same. Vol., ed.̂eds. Google Patents.
CHOI, S. H., et al., US20130045266A1 Feb. 2013. Method for preparing polymeric biomaterials having immobilized drug delivery system comprising bioactive molecules loaded particle carrier. Vol., ed.̂eds. Google Patents.
Ashammakhi, N., et al., US20060002979A1 Jan. 2006. Multifunctional biodegradable composite and surgical implant comprising said composite. Vol., ed.̂eds. Google Patents.
Shea, L., et al., US20060002978A1 Jan. 2006. Biodegradable scaffolds and uses thereof. Vol., ed.̂eds. Google Patents.
Fukushima, T., et al., US20110257094A1 Oct. 2011. Anti periodontitis-causing microorganism agent and medical or dental materials using the same. Vol., ed.̂eds. Google Patents.
Martin, D. P., Guo, K., Williams, S. F., US20140275325A1 Sep. 2014. Compositions and Devices of Poly-4-Hydroxybutyrate. Vol., ed.̂eds. Google Patents.
Elly, L., et al., WO2001035932A2 May 2001. Sustained drug delivery from structural matrices. Vol., ed.̂eds. Google Patents.
Jansen, J. A., et al., WO2006088368A2 Aug. 2006. Dna-based coatings for implants. Vol., ed.̂eds. Google Patents.
INANç, B., INANç, L., WO2011030185A1 May 2011. Cell-guiding fibroinductive and angiogenic scaffolds for periodontal tissue engineering. Vol., ed.̂eds. Google Patents.
Chung, C. P., et al., WO2012144778A9 Jan. 2013. Surface-active collagen barrier membrane to which a peptide is bound. Vol., ed.̂eds. Google Patents.

### NON-PATENT REFERENCES

1. Boateng, J. S., et al., Wound healing dressings and drug delivery systems: a review. J Pharm Sci, 2008. 97(8): p. 2892-923.
2. Kim, J. J. and G. R. Evans, Applications of biomaterials in plastic surgery. Clin Plast Surg, 2012. 39(4): p. 359-76.
3. Kruger, T. E., A. H. Miller, and J. Wang, Collagen scaffolds in bone sialoprotein-mediated bone regeneration. Scientific World Journal, 2013. 2013: p. 812718.
4. Mafi, P., et al., Evaluation of biological protein-based collagen scaffolds in cartilage and musculoskeletal tissue engineering—a systematic review of the literature. Curr Stem Cell Res Ther, 2012. 7(4): p. 302-9.
5. Taylor, B. C., et al., Induced membrane technique for reconstruction to manage bone loss. J Am Acad Orthop Surg, 2012. 20(3): p. 142-50.
6. Dimitriou, R., et al., The role of barrier membranes for guided bone regeneration and restoration of large bone defects: current experimental and clinical evidence. BMC Med, 2012. 10: p. 81.
7. Nyman, S., et al., New attachment formation by guided tissue regeneration. J Periodontal Res, 1987. 22(3): p. 252-4.
8. Gottlow, J., et al., New attachment formation in the human periodontium by guided tissue regeneration. Case reports. J Clin Periodontol, 1986. 13(6): p. 604-16.
9. Tomlin, E. M., S. J. Nelson, and J. A. Rossmann, Ridge preservation for implant therapy: a review of the literature. Open Dent J, 2014. 8: p. 66-76.
10. Donos, N., N. Mardas, and V. Chadha, Clinical outcomes of implants following lateral bone augmentation: systematic assessment of available options (barrier membranes, bone grafts, split osteotomy). J Clin Periodontol, 2008. 35(8 Suppl): p. 173-202.
11. Reynolds, M. A., et al., The efficacy of bone replacement grafts in the treatment of periodontal osseous defects. A systematic review. Ann Periodontol, 2003. 8(1): p. 227-65.
12. Froum, S. J., C. Gomez, and M. R. Breault, Current concepts of periodontal regeneration. A review of the literature. N Y State Dent J, 2002. 68(9): p. 14-22.
13. Rosenberg, E. and L. F. Rose, Biologic and clinical considerations for autografts and allografts in periodontal regeneration therapy. Dent Clin North Am, 1998. 42(3): p. 467-90.
14. Polo-Corrales, L., M. Latorre-Esteves, and J. E. Ramirez-Vick, Scaffold design for bone regeneration. J Nanosci Nanotechnol, 2014. 14(1): p. 15-56.
15. Marston, W. A., Dermagraft, a bioengineered human dermal equivalent for the treatment of chronic nonhealing diabetic foot ulcer. Expert Rev Med Devices, 2004. 1(1): p. 21-31.
16. Holmes, C., et al., Collagen-based wound dressings for the treatment of diabetes-related foot ulcers: a systematic review. Diabetes Metab Syndr Obes, 2013. 6: p. 17-29.
17. Puri, K. and N. Puri, Local drug delivery agents as adjuncts to endodontic and periodontal therapy. J Med Life, 2013. 6(4): p. 414-9.
18. Kanellakopoulou, K. and E. J. Giamarellos-Bourboulis, Carrier systems for the local delivery of antibiotics in bone infections. Drugs, 2000. 59(6): p. 1223-32.
19. Stemberger, A., et al., Local treatment of bone and soft tissue infections with the collagen-gentamicin sponge. Eur J Surg Suppl, 1997(578): p. 17-26.
20. Bessa, P. C., M. Casal, and R. L. Reis, Bone morphogenetic proteins in tissue engineering: the road from the laboratory to the clinic, part I (basic concepts). J Tissue Eng Regen Med, 2008. 2(1): p. 1-13.
21. Carreira, A. C., et al., Bone morphogenetic proteins: facts, challenges, and future perspectives. J Dent Res, 2014. 93(4): p. 335-45.
22. Froum, S., et al., The use of enamel matrix derivative in the treatment of periodontal osseous defects: a clinical decision tree based on biologic principles of regeneration. Int J Periodontics Restorative Dent, 2001. 21(5): p. 437-49.
23. Kaigler, D., et al., Platelet-derived growth factor applications in periodontal and peri-implant bone regeneration. Expert Opin Biol Ther, 2011. 11(3): p. 375-85.
24. Busch, O., et al., Guided tissue regeneration and local delivery of insulinlike growth factor I by bioerodible polyorthoester membranes in rat calvarial defects. Int J Oral Maxillofac Implants, 1996. 11(4): p. 498-505.
25. Damrongsri, D., et al., Effects of Delta12-prostaglandin J2 on bone regeneration and growth factor expression in rats. Clin Oral Implants Res, 2006. 17(1): p. 48-57.
26. Marks, S. C., Jr. and S. C. Miller, Local delivery of prostaglandin E1 induces periodontal regeneration in adult dogs. J Periodontal Res, 1994. 29(2): p. 103-8.
27. Park, J. W., et al., Bone healing with oxytocin-loaded microporous beta-TCP bone substitute in ectopic bone formation model and critical-sized osseous defect of rat. J Clin Periodontol, 2014. 41(2): p. 181-90.
28. Sarment, D. P., et al., Effect of rhPDGF-BB on bone turnover during periodontal repair. J Clin Periodontol, 2006. 33(2): p. 135-40.
29. Vandersall, D. C., Periodontics in the next millennium. Dent Clin North Am, 1998. 42(3): p. 543-60.
30. Wehrhan, F., et al., PEG matrix enables cell-mediated local BMP-2 gene delivery and increased bone formation in a porcine critical size defect model of craniofacial bone regeneration. Clin Oral Implants Res, 2012. 23(7): p. 805-13.
31. Yu, N.Y., et al., Spatial control of bone formation using a porous polymer scaffold co-delivering anabolic rhBMP-2 and anti-resorptive agents. Eur Cell Mater, 2014. 27: p. 98-109; discussion 109-111.
32. Reddy, M. S., et al., Efficacy of controlled-release subgingival chlorhexidine to enhance periodontal regeneration. J Periodontol, 2003. 74(4): p. 411-9.
33. Fallucco, M. A. and M. H. Carstens, Primary reconstruction of alveolar clefts using recombinant human bone morphogenic protein-2: clinical and radiographic outcomes. J Craniofac Surg, 2009. 20 Suppl 2: p. 1759-64.
34. Mellonig, J. T., P. Valderrama Mdel, and D. L. Cochran, Histological and clinical evaluation of recombinant human platelet-derived growth factor combined with beta tricalcium phosphate for the treatment of human Class III furcation defects. Int J Periodontics Restorative Dent, 2009. 29(2): p. 169-77.
35. Stavropoulos, A., et al., A phase IIa randomized controlled clinical and histological pilot study evaluating rhGDF-5/beta-TCP for periodontal regeneration. J Clin Periodontol, 2011. 38(11): p. 1044-54.
36. Wikesjo, U. M., et al., Space-providing expanded polytetrafluoroethylene devices define alveolar augmentation at dental implants induced by recombinant human bone morphogenetic protein 2 in an absorbable collagen sponge carrier. Clin Implant Dent Relat Res, 2003. 5(2): p. 112-23.
37. Gentile, P., et al., Polymeric membranes for guided bone regeneration. Biotechnol J, 2011. 6(10): p. 1187-97.
38. Ku, Y., et al., Chitosan/poly(L-lactic acid) multilayered membrane for guided tissue regeneration. J Biomed Mater Res A, 2009. 90(3): p. 766-72.
39. Hosokawa, R., et al., Controlled local application of basic fibroblast growth factor (FGF-2) accelerates the healing of GBR. An experimental study in beagle dogs. Clin Oral Implants Res, 2000. 11(4): p. 345-53.
40. Chang, C. Y. and S. Yamada, Evaluation of the regenerative effect of a 25% doxycycline-loaded biodegradable membrane for guided tissue regeneration. J Periodontol, 2000. 71(7): p. 1086-93.
41. Kurtis, B., et al., Effect of polylactide/glycolide (PLGA) membranes loaded with metronidazole on periodontal regeneration following guided tissue regeneration in dogs. J Periodontol, 2002. 73(7): p. 694-700.
42. Chang, A. I. and K. Appasani, Emphasizing the need to more fully understand development: a stem cells and regenerative medicine meeting report. J Tissue Eng Regen Med, 2007. 1(4): p. 274-80.
43. Lysaght, M. J. and J. Reyes, The growth of tissue engineering. Tissue Eng, 2001. 7(5): p. 485-93.
44. Teng, Y. T., J. Sodek, and C. A. McCulloch, Gingival crevicular fluid gelatinase and its relationship to periodontal disease in human subjects. J Periodontal Res, 1992. 27(5): p. 544-52.
45. De Sanctis, M., G. Zucchelli, and C. Clauser, Bacterial colonization of bioabsorbable barrier material and periodontal regeneration. J Periodontol, 1996. 67(11): p. 1193-200.
46. Golub, L. M., et al., A matrix metalloproteinase inhibitor reduces bone-type collagen degradation fragments and specific collagenases in gingival crevicular fluid during adult periodontitis. Inflamm Res, 1997. 46(8): p. 310-9.
47. Hong, M. H., et al., Drug-loaded porous spherical hydroxyapatite granules for bone regeneration. J Mater Sci Mater Med, 2011. 22(2): p. 349-55.
48. Muthukuru, M., Bone morphogenic protein-2 induces apoptosis and cytotoxicity in periodontal ligament cells. J Periodontol, 2013. 84(6): p. 829-38.
49. Muthukuru, M. and J. Sun, Doxycycline counteracts bone morphogenic protein 2-induced osteogenic mediators. J Periodontol, 2013. 84(5): p. 656-65.
50. Muthukuru, M., Technical advance: decreased helper T cells and increased natural killer cells in chronic periodontitis analyzed by a novel method for isolating resident lymphocytes. J Leukoc Biol, 2012. 92(3): p. 683-92.
51. Muthukuru, M. and C. W. Cutler, Upregulation of immunoregulatory Src homology 2 molecule containing inositol phosphatase and mononuclear cell hyporesponsiveness in oral mucosa during chronic periodontitis. Infect Immun, 2006. 74(2): p. 1431-5.
52. Muthukuru, M. and C. W. Cutler, Antigen capture of Porphyromonas gingivalis by human macrophages is enhanced but killing and antigen presentation are reduced by endotoxin tolerance. Infect Immun, 2008. 76(2): p. 477-85.
53. Muthukuru, M., R. Jotwani, and C. W. Cutler, Oral mucosal endotoxin tolerance induction in chronic periodontitis. Infect Immun, 2005. 73(2): p. 687-94.

1. The invention claimed is a composition for bioabsorbable polymeric composite comprising:
a) a hydrogel polymeric core component;
b) two fibrous polymeric peripheral components; and
c) two bioactive sites.

2. The polymeric composite of claim 1, wherein the said bioactive sites are crosslinked.

3. The polymeric composite of claim 1, wherein the two bioactive sites are independent of their crosslinked characteristics.

4. The polymeric composite of claim 1, wherein the two bioactive sites are drug delivery sites.

5. The polymeric composite of claim 1, wherein the two bioactive sites can encapsulate distinct molecules or drugs.

6. The polymeric composite of claim 1, wherein the two bioactive sites can encapsulate identical molecules or drugs.

7. The polymeric composite of claim 1, wherein the two bioactive sites can independently regulate their rates of drug release.

8. The polymeric composite of claim 1, wherein the two bioactive sites can encapsulate and deliver organic molecules, inorganic molecules, drugs, antimicrobials agents, anti-inflammatory agents, anti-collagenase agents, growth factors, genes, cells, bioactive molecules or bio-inert molecules that have wound healing and/or growth promoting properties.

9. The composite of claim 1, wherein one of the bioactive sites is polymeric core component.

10. The composite of claim 1, wherein one of the bioactive sites in one of the fibrous polymeric peripheral components.

11. The composite of claim 1, wherein polymeric hydrogel core is composed of microspheres, nanospheres or participles capable of drug delivery.

12. The composite of claim 1, wherein the two fibrous peripheral components are composed of polymeric collagen.

13. The composite of claim 1, wherein the said hydrogel component and the fibrous components have distinct mechanical and bioactive characteristics.

14. The composite of claim 1, wherein the one of the fibrous peripheral component is most extensively crosslinked component among the said components in the composite.

15. The composite of claim 1, wherein one of the fibrous peripheral components has mechanical properties of a barrier membrane.

16. The composite of claim 1, wherein the said compositions are fabricated in the form of a composite membrane.

17. The composite of claim 1, wherein drug release kinetics can be altered when the two bioactive sites encapsulate and deliver a single molecular drug.

18. The composite of claim 1, wherein additive or synergistic regenerative outcome are achieved when the two bioactive sites encapsulate and deliver distinct drugs.

19. The composite of claim 1, wherein the compositions are suitable for enhancing wound healing and tissue augmentation when applied at tissue engineering sites.

20. The composite of claim 1, wherein the compositions are suitable for guided tissue regeneration and guided bone regeneration.
